(19) United States
(12) Patent Application Publication
(10) Pub. No.: US 2025/0090780 A1
(43) Pub. Date: Mar. 20, 2025

(54) ENDOTRACHEAL TUBE WITH PRESSURE SENSOR AND A METHOD OF MANUFACTURE THEREOF

(71) Applicant: University of Connecticut, Farmington, CT (US)

(72) Inventor: Denis Lafreniere, West Hartford, CT (US)

(21) Appl. No.: 18/885,899

(22) Filed: Sep. 16, 2024

Related U.S. Application Data
(60) Provisional application No. 63/583,010, filed on Sep. 15, 2023.

(51) Int. Cl. A61M 16/04; A61M 16/00

(52) U.S. Cl. CPC A61M 16/0434; A61M 16/0003; A61M 16/026; A61M 2016/0027; A61M 2205/0216; A61M 2205/025; A61M 2205/3327; A61M 2205/3331; A61M 2205/50; A61M 2207/10; A61M 2210/1032

(57) ABSTRACT

An endotracheal tube includes a conduit having a proximal end and a distal end with a cuff located downstream of the proximal end and upstream of the distal end. The cuff lies closer to the distal end and is operative to create a seal between the conduit and an anatomical feature into which the conduit is inserted. A pressure sensor located between the cuff and the proximal end of the conduit is operative to measure a pressure imposed on the anatomical feature by the conduit.

### CROSS-REFERENCE TO RELATED APPLICATIONS

This application claims priority to U.S. non-provisional application having Ser. No. 63/583,010 filed on Sep. 15, 2023, the entire contents of which are hereby incorporated in their entirety.

### BACKGROUND OF THE INVENTION

### 1. Field of the Invention

This disclosure relates to medical devices, and in particular to endotracheal tubes. In particular, this disclosure relates to endotracheal tubes with pressure sensors and to a method of manufacture thereof.

### 2. Description of the Related Art

Endotracheal tubes (ETTs) are used routinely in the operating room, the emergency room and intensive care unit (ICU) settings. Some patients in the ICU need an ETT for an extended period as they are on ventilator support. Current practice recommends that a patient with an ETT inserted for more than 10 days should be strongly considered to receive a temporary tracheostomy to avoid long term complications from the endotracheal tube. These complications can also occur in patients even when the endotracheal tube is used for shorter durations. Prolonged intubation can lead to tracheal injury, including ulceration, stenosis, and granuloma formation, which may result in difficulty breathing or the need for further surgical intervention. Patients who develop scarring of the posterior larynx may require additional surgery which may result in the patient having a hoarse voice or need for a permanent tracheotomy.

It is therefore desirable to develop a device and methodologies that predict when complications associated with the use of endotracheal tubes will arise so that steps can be taken to prevent or minimize the aforementioned complications.

### SUMMARY

An endotracheal tube includes a conduit having a proximal end and a distal end with a cuff located downstream of the proximal end and upstream of the distal end. The cuff lies closer to the distal end and is operative to create a seal between the conduit and an anatomical feature into which the conduit is inserted. A pressure sensor located between the cuff and the proximal end of the conduit is operative to measure a pressure imposed on the anatomical feature by the conduit.

A method of manufacturing a conduit includes disposing a pressure sensor onto an endotracheal tube. The endotracheal tube includes a conduit having a proximal end and a distal end with a cuff located downstream of the proximal end and upstream of the distal end. The cuff lies closer to the distal end and is operative to create a seal between the conduit and an anatomical feature into which the conduit is inserted. A pressure sensor located between the cuff and the proximal end of the conduit is operative to measure a pressure imposed on the anatomical feature by the conduit. Data pertaining to the measured pressure is transmitted to a recording device.

A system for monitoring the health of a patient while intubated includes an endotracheal tube with a pressure sensor attached thereto. The endotracheal tube includes a conduit having a proximal end and a distal end with a cuff located downstream of the proximal end and upstream of the distal end. The cuff lies closer to the distal end and is operative to create a seal between the conduit and an anatomical feature into which the conduit is inserted. A pressure sensor located between the cuff and the proximal end of the conduit is operative to measure a pressure imposed on the anatomical feature by the conduit. Data pertaining to the measured pressure is transmitted to a monitoring system. The monitoring system receives output of the pressure sensor and compares the output as monitoring data to medical data. The comparison is used for predicting a pathological condition caused by use of the endotracheal tube.

### BRIEF DESCRIPTION OF THE FIGURES

FIG. 1A is a depiction of the laryngeal anatomy;

FIG. 1B is a depiction of a patient intubated with a conventional endotracheal tube that does not have a pressure sensor;

FIG. 2 is a schematic depiction of an exemplary endotracheal tube that contacts a posterior region of the larynx through a pressure sensor;

FIGS. 3-6 depict various configurations of the pressure sensor on the conduit of the endotracheal tube;

FIG. 7 is a graph that shows average pressures in centimeters of H₂O for 6, 7, and 8 millimeter outer diameter endotracheal tubes with an attached piezoresitive sensor placed over the posterior glottis in a laryngeal model.

### DETAILED DESCRIPTION

Disclosed herein is an endotracheal tube with an inbuilt pressure sensor that measures the pressure on the posterior portion of the larynx when the endotracheal tube is deployed. The pressure sensor advantageously permits monitoring of the pressure exerted by an endotracheal tube (ETT) on the larynx at a region proximal to a cuff. The pressure sensor contacts the endotracheal tube proximal to the cuff in a region where the endotracheal tube would normally contact the posterior part of the larynx when deployed. If a pressure greater than the perfusion pressure of 25 centimeters of water is detected by the pressure sensor, then one or more corrective procedures may need to be performed. Corrective procedures may include, deploying an endotracheal tube having a different stiffness, deploying an endotracheal tube having a smaller diameter, modification of patient position, the performance of an early tracheostomy, or a combination thereof. As a result, practitioners may use the endotracheal tubes with limited concern for collateral damage to the patient.

The use of a pressure sensor in conjunction with the endotracheal tube is advantageous because data collected from the pressure sensor may be tracked and used to estimate overall exposure and risk of scarring or other forms of tracheal injury listed above such as, for example, ulceration, stenosis and granuloma formation, which may all be minimized or avoided.

In an embodiment, the pressure sensor provides data that is descriptive of pressure between anatomical features and the endotracheal tube. Accordingly, an endotracheal tube outfitted with appropriate sensors (such as with at least one pressure sensor) may be used to alert the practitioner when the larynx is at risk due to higher than tolerable pressure. That is, embodiments of endotracheal tube according to the teachings herein (generally referred to as a “wired endotracheal tube”) may be used to provide data that is descriptive of pressure between anatomical features and the endotracheal tube.

When used in conjunction with appropriately configured processing, the wired endotracheal tube may be used to alert practitioners to potential for scarring. In some embodiments, the endotracheal tube is outfitted with a piezoresistive sensor imbedded on the posterior aspect of the tube (the area that will contact the posterior glottis). The sensor is positioned at a distance of about 1 cm proximal to the endotracheal tube cuff and extending several centimeters proximally (towards the proximal end) so it will cover the posterior glottis region.

In this non-limiting embodiment, the sensor is composed of three components, a polymer film, conductive material, and electrical leads. As force is applied to the sensing material the conductive material undergoes deformation which alters its electrical resistance. The electrical leads are attached to a processor which converts the resistance change into an electrical signal. The electrical signal may be interpreted in a number of ways. For example, the electrical signal may be converted to pressure as centimeters of water.

In some embodiments, the processor provides practitioners with an instantaneous measurement of pressure between the endotracheal tube and the surrounding anatomy. In some other embodiments, the processor periodically measures pressure. In these embodiments, the processor may output to practitioners integrated pressure (e.g., as a graph of pressure over time). In some embodiments, the processor is configured to alert practitioners on the basis of, for example, indications from historical data, a predetermined set-point, intelligence from machine learning and/or according to other guidance. A variety of techniques for data collection and display may be used.

Appropriate electronics may be used to power the wired endotracheal tube and communicate data signals, and send signals to a processor. Examples of processing suited for use with the data may be collected infrequently, periodically, near real-time and/or on a real-time basis. Generally, “real-time” is considered to refer to a temporal basis that provides for monitoring of conditions as changes occur, thus enabling mitigation of risk as it is realized.

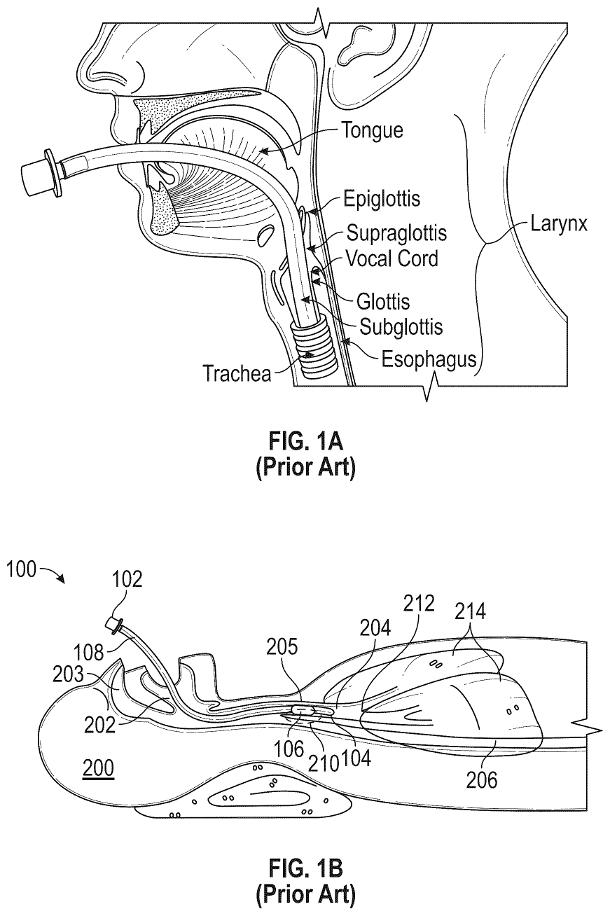

FIG. 1A is a depiction of the laryngeal anatomy. As shown in FIG. 1A, the laryngeal anatomy leads to the trachea through a narrow passageway that involves vocal cords, glottis and the sub-glottis. As can be seen, placing an endotracheal tube through the larynx and into the trachea can be challenging and risks damage to the anatomical features. Aspects of the invasive nature of an endotracheal tube are shown in FIG. 1B.

FIG. 1B is a depiction of a patient 200 intubated with a conventional endotracheal tube 100. Conventional endotracheal tubes refer to those not fitted with pressure sensors as will be detailed herein. FIG. 1B depicts the location where injury occurs to a patient 200 that is intubated with the endotracheal tube 100. The endotracheal tube 100 comprises a tube having a proximal end 102 and a distal end 104. The proximal end 102 remains outside the patient's mouth or nose and is closest to the physician performing the corrective action. It is equipped with a connector that is in fluid communication with various ventilation devices (not shown), such as a bag-valve-mask (BVM), a mechanical ventilator, and/or an anesthesia machine. Medical personnel can administer gases, control the airway, and monitor respiratory parameters through the proximal end 102.

The distal end 104 is inserted behind the tongue 202 into the trachea 204 through the vocal cords (not shown) and positioned above the carina 212, the point where the trachea 204 divides into the two main bronchi. The distal end 104 typically features an inflatable cuff 106. Once the endotracheal tube 100 is in place, this cuff 106 is inflated to create a seal between the tube 108 and the tracheal walls—the anterior wall 205 and the posterior wall 207 as seen in FIG. 1B. This prevents air from leaking around the tube 108 and also prevents aspiration of secretions into the lungs 214. The distal end 104 opening delivers oxygen and anesthetic gases directly into the trachea 204 and lungs 214, facilitating effective ventilation.

During intubation, the tube 108 is bent in order to travel from the mouth 202 or nose 203 to the trachea 204. The bending of the tube 108 promotes contact between the outer surface of the tube and the posterior wall 207 of the larynx at region 210. The continual pressure imposed by the endotracheal tube on the posterior wall of the larynx at region 210 produces some of the problems discussed above such as, for example, ulceration, stenosis, and granuloma formation.

In current practice, trauma caused by use of an endotracheal tube is sometimes not immediately recognized as patients are usually stable for several weeks to months after the endotracheal tube is removed. Patients slowly develop scarring of the posterior larynx which leads to difficulty breathing. The latent development of post-endotracheal tube induced scarring often requires additional surgery often including tracheostomy. This scarring may ultimately require a destructive procedure where portions of a vocal cord are removed to create an airway which will allow the patient to breathe adequately. This usually results in a permanent hoarse voice.

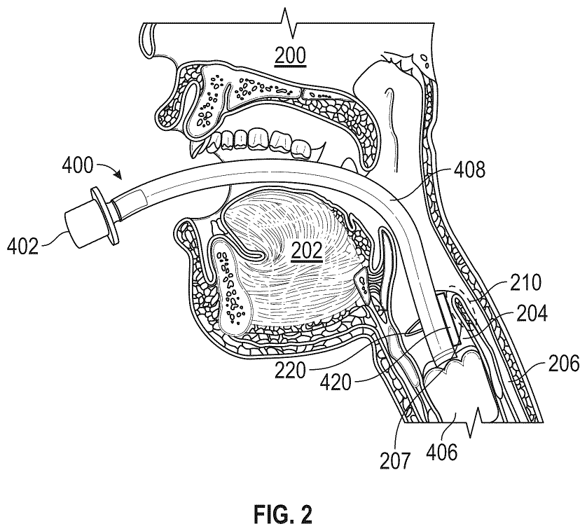

FIG. 2 is a schematic depiction of an exemplary endotracheal tube 400 that contacts a posterior region 207 of the larynx 204 through a pressure sensor 420. The endotracheal tube 400 comprises a proximal end 402 that is in fluid communication with a conduit 408. The conduit 408 is in fluid communication with the distal end (not shown) that as described in the FIG. 1B is positioned above the carina, the point where the trachea divides into the two main bronchi. The proximal end 402 and the distal end (not shown) function in the same manner as detailed above in the FIG. 1B. The proximal end 402 may contact some of the same devices (not shown) such as the bag-valve-mask (BVM), the mechanical ventilator, and/or the anesthesia machine listed above.

The pressure sensor 420 measures the pressure exerted by the endotracheal tube 408 on the posterior region 207 of the larynx 204. The pressure sensor 420 generally lies between the cuff 406 and the proximal end 402 of the endotracheal tube 400 and is generally located closer to the cuff 406 than the proximal end 402. It extends from slightly above the cuff 406 towards the proximal end 402 of the conduit 408.

In an embodiment, the pressure sensor 420 is located on the endotracheal tube at a distance of about 1 centimeter from the cuff and extends towards the proximal end 402 past the point at which the endotracheal tube contacts the larynx 204. As will be detailed below, the pressure sensor contacts the outer circumferential surface of the conduit 408 and typically lies between the outer circumferential surface of the conduit 408 and the posterior region 207 of the larynx 204.

The pressure sensor 420 that may be used includes, without limitation, some embodiments of thin film sensors, thick film sensors, piezoelectric sensor, a strain gauge sensor, a capacitive sensor or a piezoresistive sensor. Thin-film sensors are based on the same principle as strain gauges, which are grid-type resistance structures whose geometric stretching and compression result in a measurable resistance change due to length and thickness differences induced. For a thin-film sensor, four resistors are arranged on a diaphragm in the form of a Wheatstone bridge to detect the deformation of the diaphragm under pressure. In the ‘thin-film process’, these strain gauges are attached onto a (e.g. metallic) base element and structured (sputtering with associated photolithography and etching). An example of a thin-film sensor includes DFRobot RP Series Thin Film Pressure Sensors. These sensors are made of ultra-thin film and exhibit good mechanical properties.

These sensors offer durability and are designed to sense static and dynamic pressure in high response speed. The RP sensors come with thin film and pressure-sensitive layer on the upper layer and thin film and conductive circuit on the lower layer. These layers in the sensors are glued together with double-sided tape. The RP sensors convert pressure into resistance by connecting the disconnected circuit of the lower layer through a pressure-sensitive layer of the upper layer. The RP sensors are available in RP-C18.3-LT, RP-C7.6-LT, RP-L-170, RP-S40-ST, RP-L-400, and RP-C7.6-ST variants.

Thick-film sensors, like thin-film sensors, use four resistors grouped to form a Wheatstone bridge. The resistance structures are “printed” onto a base element (e.g. ceramic base) using thick-film technology, and afterwards they are burnt-in at high temperature. The resistance change here is also due to the deformation of the diaphragm, resulting from the geometrical change caused by the stretching and compression of the material.

Piezoelectric sensors use materials like quartz or polyvinylidene fluoride that generate an electrical charge when subjected to pressure. The generated voltage is proportional to the applied pressure.

In strain gauge sensors, a pressure changes cause a deformation in the strain gauge, altering its electrical resistance. This change in resistance is converted into a voltage signal.

In capacitive sensors, a pressure causes changes in the distance between two capacitor plates, affecting the capacitance, which is then converted into an electrical signal.

Piezoresistive sensors utilize a semiconductor (silicon) measuring diaphragm with selectively diffused structures. They use the piezoresistive effect, which is based on the change in electrical resistance in the semiconductor materials caused by the stretching and compression, which affects the mobility of the electrons under the mechanical stress. Examples of piezoresistive sensors and related components are available from TE Connectivity of Berwyn, PA.

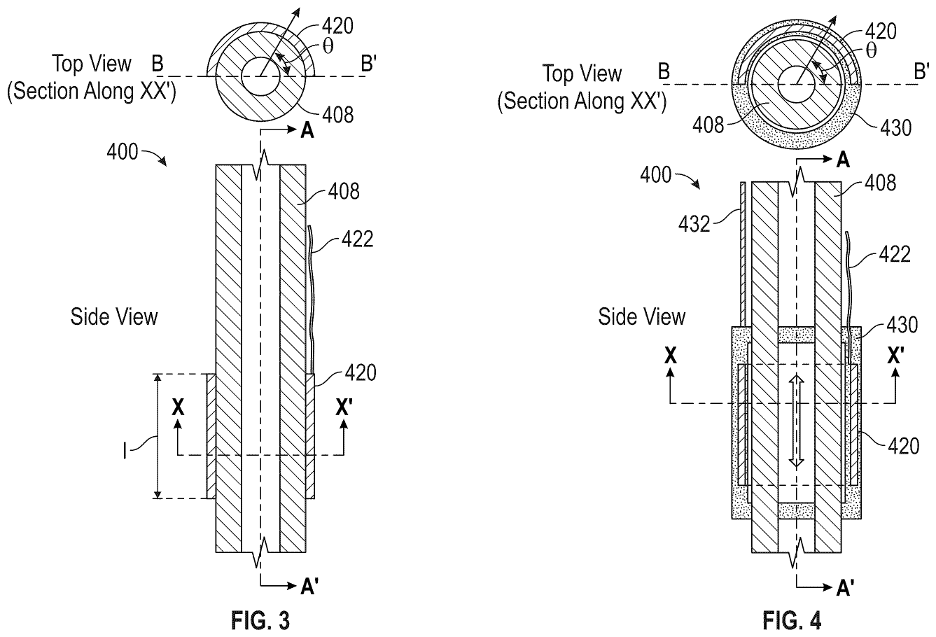

FIGS. 3-6 depict various configurations of the pressure sensor 420 on the conduit 408 of the endotracheal tube 400. In the FIGS. 3-6, only portions of the endotracheal tube 400 that contain the pressure sensor 420 are shown. Each of FIGS. 3-6 depicts a side view of the endotracheal tube in a particular configuration while the top view is obtained from Section XX′ of the respective side view.

FIG. 3 depicts an embodiment where the pressure sensor 420 may be located on an outer surface of the conduit 408 between the cuff 406 and the proximal end 402 of the endotracheal tube 400. In one embodiment, the pressure sensor 420 may be bonded to the outer surface of the conduit 408 through a layer of adhesive (not shown). The adhesive preferably comprises a biocompatible polymer such as a cyanoacrylate, a fibrin sealant (e.g., derived from human fibrinogen and thrombin), gelatin-resorcinol-formaldehyde (GRF) glue, polyethylene glycol hydrogels, albumin-glutaraldehyde adhesives, chitosan adhesives, or a combination thereof. The adhesive may be cured using radiation (e.g., UV or microwave radiation) or thermal energy.

In another embodiment, the pressure sensor 420 may be taped to the conduit 408 using adhesive tape (not shown) that is biocompatible. Both the adhesive used on the tape and the tape are preferably biocompatible. The tape may be applied to at least one surface (either an inner surface or an outer surface) of the pressure sensor and facilitates bonding of the pressure sensor to the conduit. The inner surface of the pressure sensor 420 is the surface that faces the conduit 408 while the outer surface is that surface that contacts the surface of the larynx. The tape may surround the pressure sensor 420 and the larynx. Examples of biocompatible adhesive tape include silicone medical tape, paper medical tape, polyurethane film tape, hydrocolloid tape, elastic adhesive tape, zinc oxide adhesive tape and acrylic-based medical tape.

The conduit 408 preferably is manufactured from a biocompatible, flexible material such as, for example, a polysiloxane (e.g., silicone elastomer), a polyolefin (e.g., polyethylene, polypropylene, or a combination thereof), a fluoropolymers, polyvinylchloride, polyurethane, or a combination thereof. The conduit is preferably an elastomer comprising at least one of the foregoing polymers. The elastomer is selected for its ability to minimize trauma to the airway during intubation, especially for short-and long-term use

In an embodiment, the conduit 408 may be coated with a layer of polytetrafluoroethylene (TEFLON) to render the conduit 408 biocompatible. The polytetrafluoroethylene coating minimizes friction and sticking during insertion, making intubation easier and potentially safer.

In an embodiment, the elastic modulus of the elastomer used in the endotracheal tube is 0.1 to 600 MPa, preferably 1 to 300 MPa, and more preferably 2 to 50 MPa measured as per ASTM D 638. If the pressure sensor begins to record pressures greater than 25 centimeters of water, then a softer elastomer may be used to replace a harder endotracheal tube. For example, a silicone elastomer that has an elastic modulus of 0.1 to 5 MPa may be used to replace a polyurethane elastomer having an elastic modulus of 10 to 50 MPa, when the pressure sensor records pressures on the larynx of greater than 25 centimeters of water.

The conduit 408 has an outer diameter of 4 to 12 millimeters, preferably 5 to 10 millimeters with a wall thickness of 0.5 to 4 millimeters, preferably 1 to 3 millimeters.

The pressure sensor 420 covers a sufficient area of the circumferential surface of the conduit so that it can easily be located at a region within the trachea where it will record the pressure on the posterior portion of the larynx. In an embodiment, the pressure sensor 420 may be of a length effective to always contact the inner surface of the posterior wall of the larynx at a region of maximum pressure irrespective of the location of the conduit 408 in the larynx. As noted above, the pressure sensor 420 typically begins at about 1 centimeter above the cuff and extends towards the proximal end of the endotracheal tube. It typically is of a length that permits recording the pressure applied by the conduit 408 on the posterior portion of the larynx. In an embodiment, the pressure sensor 420 may lie on an outer surface of the conduit 408 and be in direct contact with an inner surface of the larynx.

With reference to the side view of the FIG. 3, the pressure sensor 420 extends over the surface of the conduit 408 for a length “1”. The length 1 is measured parallel to the longitudinal axis XX′ of the conduit 408. The length of the pressure sensor may be selected so that it contacts the region 210 located at the posterior region 207 of the larynx 204 without significant manipulation or positional adjustment of the endotracheal tube. In an embodiment, the length of the pressure sensor is 30 to 60 millimeters.

As may be seen in the top view “of the FIG. 3, the pressure sensor 420 contacts the outer surface of the conduit 408 along a substantial portion of its outer circumference. The pressure sensor 420 is located on the outer surface of the conduit 408 in a region where it will easily contact the tissue in the larynx that the endotracheal tube will impinge on during intubation. In a preferred embodiment, the pressure sensor 420 contacts the surface of the conduit over a radial angle θ of 30 degrees to 270 degrees, preferably 50 to 200 degrees, and more preferably 60 to 180 degrees. In other words, lines drawn from the longitudinal axis of the conduit to the edges of the pressure sensor would include (subtend) a radial angle of 30 to 270 degrees at the center of the conduit. The radial angle is measured at the center of the conduit 408 in a plane that is perpendicular to the longitudinal axis AA′.

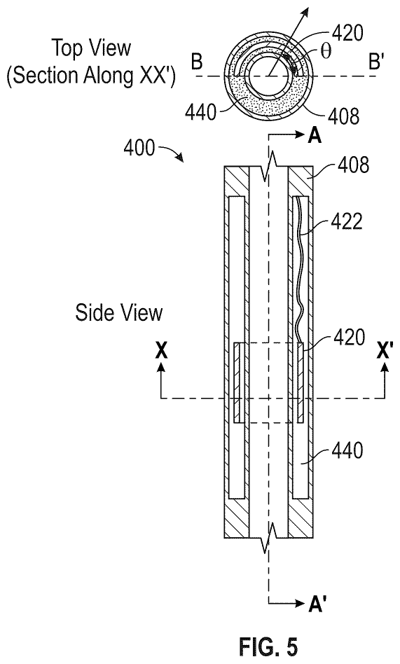

In an embodiment, the pressure sensor 420 may be in operative communication with a microprocessor or computer via electrical wires 422. The microprocessor or computer may record information received from the pressure sensor and convert it into readable data. In another embodiment, the pressure sensor may be in operative communication with the microprocessor or computer via selected electromagnetic frequencies in the radiofrequency, microwave or visible light regimes. When visible light is deployed, optical fibers may be used to transmit information from the pressure sensor to a microprocessor or computer. The electrical wires or the optical fibers may lie outside the conduit 408 as seen in FIG. 3 or they may be embedded in the wall of the conduit 408 (See FIG. 5).

In another embodiment depicted in the FIG. 4, the pressure sensor 420 may be embedded in a slider 430 that can be moved along the length of the conduit 408. The slider 430 may be displaced along the length of the conduit 408 from the distal end to the proximal end using the electrical wires 422 that contact the pressure sensor 420. Optical fibers may also be used (in lieu of electrical wires) to displace the slider 430 along the conduit 408. In another embodiment, a ductile wire 432 of limited stiffness may be used to move the slider so as to enable the pressure sensor to make contact with the posterior portion of the trachea. The ductile wire 432 is generally manufactured from a flexible polymer that has a stiffness effective to facilitate movement of the slider without damaging tissues in the laryngeal region.

The FIG. 4 also depicts a side view of the endotracheal tube 400 and a side view of a section of the conduit 408 taken at section XX′. The slider 430 has walls that are of a thickness sufficient to embed the pressure sensor 420. The sensor 420 is in electrical communication with a microprocessor or a computer via electrical wires 422, optical fibers (not shown), or via selected electromagnetic frequencies.

The slider 430 is concentrically mounted about the conduit 408 and can be transported back and forth with a minimal amount of friction. In the FIG. 4, the area of the pressure sensor 420 (e.g., its length and circumferential coverage) is selected so that it will contact the posterior portion of the larynx (through the slide) with a minimum amount of manipulation and movement. The slider would be very thin so as to not increase the outer diameter of the endotracheal tube to any significant degree. These length and angle θ are previously detailed in the FIG. 3 and will not be repeated in the interests of brevity.

The slider 430 is generally manufactured from the same list of elastomers detailed above. The slider 430 is preferably coated with a layer of polytetrafluoroethylene (TEFLON) to minimize friction with the conduit during its travel along the conduit.

FIG. 5 depicts another embodiment of an endotracheal tube 400 where the pressure sensor 420 is located and transported in a cavity 440 in the wall of the conduit 408. The cavity 440 can be located in the entire conduit 408 or alternatively, in only a section of the conduit 408 that is proximate to the cuff 406. The cavity is of a length that is effective to permit the pressure sensor to contact the region where the endotracheal tube 400 contacts the posterior portion of the larynx in region 210.

If the cavity 440 extends the entire length of the conduit 408, then the pressure sensor 420 can be moved into the proper position pneumatically (using pressurized air or using a fluid). Alternatively, the pressure sensor 420 can be moved through the cavity along the length of the conduit 408 via a ductile wire 432 (see FIG. 4). The dimensions of the pressure sensor (i.e., the length and the angular coverage) are similar to those described in the FIG. 3 and will not be repeated here.

In an embodiment, in one manner of using the endotracheal tube of the FIGS. 3, 4 and 5, the endotracheal tube may be inserted into the laryngeal region via the mouth or the nose till the distal end enters through the trachea via the vocal cords, the glottis and the sub-glottis. The cuff is pressurized to create a seal between the tube and the create a seal between the tube and the tracheal walls. The position of the endotracheal tube is adjusted so that the pressure sensor contacts the posterior wall of the trachea. In the case of the endotracheal tube of the FIG. 3 this adjustment of position may be conducted by physically manipulating the proximal end of the endotracheal tube.

In the case of the endotracheal tube of the FIG. 4, the position of the slider (containing the pressure sensor) may be adjusted using the electrical wires, a fiber optical cable or the ductile rod 432. In this case, the slider may be initially located adjacent to the cuff 406 and contacting the cuff. Upon inflating the cuff to create the seal, the slider may be moved away from the cuff (towards the proximal end) to the region where the conduit contacts the posterior portion of the larynx. It may be deployed between the conduit and the posterior portion of the larynx where it may be used to continuously monitor pressure.

In the case of the endotracheal tube of the FIG. 5, the position of the pressure sensor may be adjusted after the cuff is inflated using a pressurized fluid (e.g., air or nitrogen) or a ductile wire or rod (as previously described with regard to FIG. 4). The pressure sensor may initially be located closer to the proximal end of the conduit 408. Upon inserting the endotracheal tube and inflating the cuff, a pressurized fluid may be used to move the pressure sensor in the cavity towards the cuff from the proximal end. The pressure sensor is moved to the region where the conduit contacts the posterior portion of the trachea. It may be deployed between the conduit and the posterior portion of the trachea where it may be used to continuously monitor pressure.

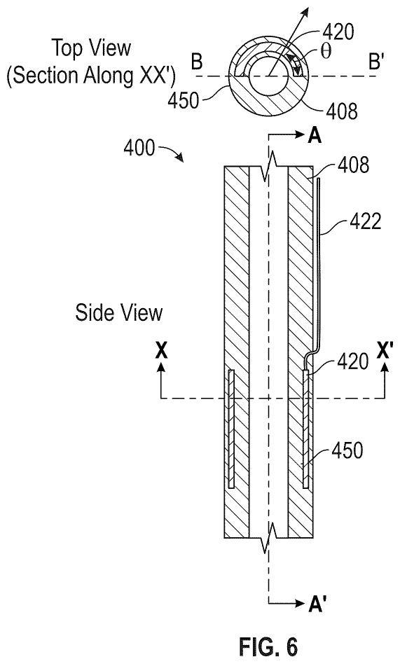

FIG. 6 depicts another embodiment, where an outer portion of the conduit 408 is slit circumferentially as well as parallel to the length of the conduit to obtain a sliver 450 of the conduit. The sliver 450 is typically less than 1.5 millimeters, preferably less than 1 millimeter thick. In other words, an outer sliver 450 of the conduit is removed to produce a pocket into which the pressure sensor 420 is disposed. The sliver 450 is then adhesively bonded back to the portion of the conduit 408 from which it was previously separated. The sliver 450 therefore behaves as a sheath which protects the pressure sensor 420 from damage. The sheath (which is identical to the sliver 450) and the pressure sensor are both thin enough to not promote any protrusions on the conduit 408. They are also thin enough to be sensitive to variations in pressure when a patient is intubated with the endotracheal tube 400.

Upon deploying the endotracheal tube in the manner described above, the pressure sensor monitors the pressure continuously and feeds data back to the microprocessor or computer where the data is monitored. If the pressure increases above 25 centimeters of water, then the position of the patient may be modified, such as with a head tuck. Other interventions can include endotracheal tube removal and replacement of the conduit with a new softer conduit. The process may be repeated till a satisfactory operating pressure is reached. Alternatively, corrective procedures such as the performance of an early tracheostomy may be conducted.

In summary, the endotracheal tube with a contact imbedded pressure sensor may be advantageously used to measure the pressure generated by the tube onto the posterior inter-arytenoid tissues of the larynx. This tube with sensor will provide information regarding the degree of pressure on the posterior larynx which will help guide the providers decision regarding the need for early tracheostomy. This device will help reduce the number of posterior glottic stenosis cases as endotracheal tubes will be removed before they can cause harm to the larynx.

In one embodiment, the endotracheal tube with the affixed embedded pressure sensor may be manufactured by disposing a pressure sensor onto an endotracheal tube. The endotracheal tube comprises a conduit having a proximal end and a distal end and a cuff located downstream of the proximal end and upstream of the distal end. The cuff lies closer to the distal end and is operative to create a seal between the conduit and an anatomical feature into which the conduit is inserted. The pressure sensor is disposed onto the endotracheal tube between the cuff and the proximal end of the conduit. The pressure sensor is operative to measure a pressure imposed on the anatomical feature by the conduit and transmit data about the pressure to a recording device. The recording device is a processor (e.g., a microprocessor or a computer) and provides a physician with a readable output of the pressure imposed by the endotracheal tube on the posterior region of the larynx.

In an embodiment, the pressure sensor is embedded beneath the outer surface of the conduit (i.e., the outer surface of the conduit acts as a protective sheath for the pressure sensor) or embedded in a slider that is operative to move along the length of the conduit. In yet another embodiment, the pressure sensor is embedded in a cavity in the walls of the conduit.

In an embodiment, the endotracheal tube with the affixed pressure sensor can be a part of a system for monitoring health of a patient while intubated. The system comprises the endotracheal tube that comprises a conduit having a proximal end and a distal end with a cuff located downstream of the proximal end and upstream of the distal end. The cuff lies closer to the distal end and is operative to create a seal between the conduit and an anatomical feature into which the conduit is inserted. The pressure sensor is located between the cuff and the proximal end of the conduit, where the pressure sensor is operative to measure a pressure imposed on the anatomical feature by the conduit. The pressure sensor is in operative communication with a monitoring system for receiving output of the pressure sensor. The monitoring system is operative to compare presently obtained output (e.g., monitoring medical data) to previously obtained medical data. This comparison may be used for predicting a pathological condition caused by use of the endotracheal tube. Operative communication as used herein is inclusive of electrical communication, optical communication, radiofrequency or microwave frequency communication, or a combination thereof.

In an embodiment, the medical data obtained may be used to adjust an orientation of the endotracheal tube within the patient. The data obtained may be used to predict whether the particular patient may suffer from a pathological condition. In another embodiment, the receiving of medical data, the comparing of such medical data with previously obtained medical data and the predicting of a pathological condition may be provided on an ongoing basis so long as the patient is intubated.

The endotracheal tube together with the appended pressure sensor is exemplified by the following non-limiting example.

### Example

This example is conducted to demonstrate the use of endotracheal tubes of varying sizes and the pressure exerted by tubes of different sizes on the larynx. Three endotracheal tubes having an outer diameter of 6, 7, and 8 mm respectively were modified with a piezoelectric pressure sensor. These endotracheal tubes were then used to intubate a laryngeal model. Pressures were measured at a hypopharyngeal laryngeal angle (HLA) of 90 degrees and the laryngeal platform was raised in 2 millimeter increments to create a more acute HLA ranging from 90 to 45 degrees. These same endotracheal tubes were also used to intubate the larynx of a fresh frozen cadaver with pressure measurements made with the head in neutral and flexed positions. Pressure measurements were converted into centimeters of H₂O.

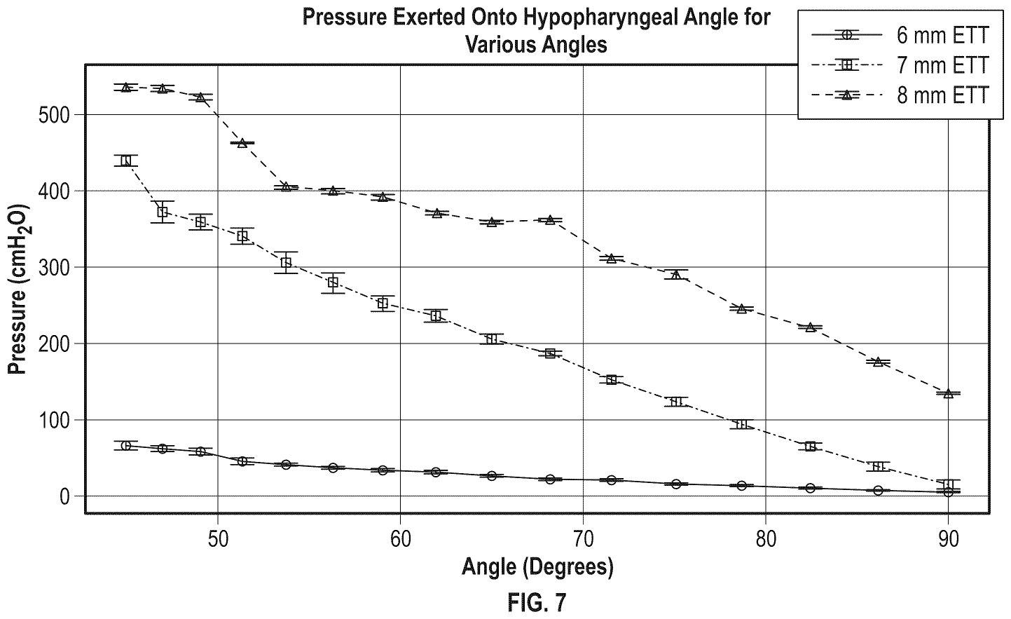

FIG. 7 is a graph that shows average pressures in centimeters of H₂O for 6, 7, and 8 millimeter outer diameter endotracheal tubes with an attached piezoresistive sensor placed over the posterior glottis in a laryngeal model with increasing anterior displacement of the larynx creating a hypopharyngeal laryngeal angle ranging from 90 to 45 degrees. From the FIG. 7 it may be seen that pressure on the larynx is reduced with the decrease in outer diameter.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112 (f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

While the invention has been described with reference to some embodiments, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiments disclosed as the best mode contemplated for carrying out this invention, but that the invention will include all embodiments falling within the scope of the appended claims.

What is claimed is:

1. An endotracheal tube comprising:
a conduit having a proximal end and a distal end;
a cuff located downstream of the proximal end and upstream of the distal end; where the cuff lies closer to the distal end and is operative to create a seal between the conduit and an anatomical feature into which the conduit is inserted; and
a pressure sensor located between the cuff and the proximal end of the conduit; where the pressure sensor is operative to measure a pressure imposed on the anatomical feature by the conduit.

2. The endotracheal tube of claim 1, where the anatomical feature is a trachea.

3. The endotracheal tube of claim 1, where the pressure sensor contacts an outer circumferential surface of the conduit.

4. The endotracheal tube of claim 1, where the pressure sensor extends from at least one centimeter from the cuff towards the proximal end of the conduit and at least contacts the anatomical feature that is pressured by contact with the conduit.

5. The endotracheal tube of claim 1, where the pressure sensor contacts an outer circumferential surface of the conduit and subtends a radial angle of 30 to 270 degrees at a longitudinal axis of the conduit.

6. The endotracheal tube of claim 1, where the pressure sensor comprises a thin film sensor, a thick film sensor, a piezoelectric sensor, a strain gauge sensor, a capacitive sensor or a piezoresistive sensor.

7. The endotracheal tube of claim 1, where the pressure sensor is a piezoresistive sensor.

8. The endotracheal tube of claim 1, where the conduit comprises an elastomer.

9. The endotracheal tube of claim 1, where the elastomer comprises a polysiloxane, a polyolefin, a fluoropolymer, a polyvinylchloride, a polyurethane, or a combination thereof.

10. The endotracheal tube of claim 1, where the pressure sensor contacts the conduit via an adhesive.

11. The endotracheal tube of claim 1, where the adhesive is biocompatible.

12. The endotracheal tube of claim 1, where the pressure sensor is mounted in a slider that traverses a surface of the conduit.

13. The endotracheal tube of claim 1, where the pressure sensor is located in a cavity in a wall of the conduit.

14. The endotracheal tube of claim 1, where an adhesive tape bonds the pressure sensor to the conduit.

15. A method of manufacturing a conduit, the method comprising:
disposing a pressure sensor onto an endotracheal tube;
where the endotracheal tube comprises:
a conduit having a proximal end and a distal end;
a cuff located downstream of the proximal end and upstream of the distal end; where the cuff lies closer to the distal end and is operative to create a seal between the conduit and an anatomical feature into which the conduit is inserted; and
where the pressure sensor is disposed onto the endotracheal tube between the cuff and the proximal end of the conduit; where the pressure sensor is operative to measure a pressure imposed on the anatomical feature by the conduit and transmit data about the pressure to a recording device.

16. The method of claim 15, where the disposing comprises one of embedding the pressure sensor within material forming the endotracheal tube or mounting the pressure sensor onto an outer surface of the endotracheal tube.

17. A system for monitoring health of a patient while intubated, the system comprising:
an endotracheal tube comprising a conduit having a proximal end and a distal end; a cuff located downstream of the proximal end and upstream of the distal end; where the cuff lies closer to the distal end and is operative to create a seal between the conduit and an anatomical feature into which the conduit is inserted; and a pressure sensor located between the cuff and the proximal end of the conduit; where the pressure sensor is operative to measure a pressure imposed on the anatomical feature by the conduit; and,
a monitoring system for receiving output of the pressure sensor and comparing the output as monitoring data to medical data for predicting a pathological condition caused by use of the endotracheal tube.

18. The system as in claim 17, further comprising:
selecting the medical data according to an orientation of the endotracheal tube within the patient.

19. The system as in claim 17, further comprising using the comparison to avert the pathological condition.

20. The system as in claim 17, wherein at least one of the receiving output, comparing and predicting is provided on an ongoing basis.
